(19) United States
(12) Patent Application Publication
(10) Pub. No.: US 2002/0182204 A1
(43) Pub. Date: Dec. 5, 2002

(54) Combination of a taxane and a cyclin-dependent kinase

(76) Inventor: Marie-Christine Bissery, Vitry Sur Seine (FR)

Correspondence Address: Finnegan, Henderson, Farabow,, Garrett & Dunner, L.L.P., 1300 I Street, N.W., Washington, DC, 20005-3315, US

(21) Appl. No.: 10/101,947

(22) Filed: Mar. 21, 2002

Related U.S. Application Data
(60) Provisional application No. 60/277,948, filed on Mar. 23, 2001.
(60) Provisional application No. 60/302,692, filed on Jul. 5, 2001.
(60) Provisional application No. 60/334,916, filed on Dec. 4, 2001.

(51) Int. Cl. A61K 38/48; A61K 38/43

(52) U.S. Cl. 424/94.63; 424/94.1

(57) ABSTRACT

The invention relates to a pharmaceutical combination comprised of paclitaxel (Taxol®), docetaxel (Taxotere®), or derivatives of them, and a cyclin-dependent kinase inhibitor. It also relates to a method of administration of such a combination, where the taxane is given intermittently and the cyclin-dependent kinase is given repeatedly within the same cycle.

### CROSS-REFERENCE TO RELATED APPLICATIONS

This application relies on, and claims the benefit of, U.S. Provisional Application No. 60/277,948, filed Mar. 23, 2001, U.S. Provisional Application No. 60/302,692, filed Jul. 5, 2001, and U.S. Provisional Application No. 60/334,916, filed Dec. 4, 2001, the disclosures of all of which are hereby incorporated by reference.

### DESCRIPTION OF THE INVENTION

1. Field of the Invention

The present invention relates to combinations of Taxol®, Taxotere®, and their analogues and other compounds that are therapeutically useful in the treatment of neoplastic diseases. More especially, the invention relates to combinations of Taxol®, Taxotere®, and their analogues with anti-tumor agents, such as cyclin-dependent kinase inhibitors.

2. Background of the Invention

Taxanes and taxoids constitute a family of naturally occurring diterpene compounds, including a potent antitumor drug, paclitaxel. Paclitaxel (Taxol®), originally isolated from the bark of the Pacific Yew tree (Taxus brevifolia), has been shown to be highly effective in adjuvant and neo-adjuvant therapies for patients with breast and ovarian cancers. More recently, its semisynthetic analogue, docetaxel (Taxotere®), has also been found effective in breast cancer chemotherapy. The diseases sensitive to this class of antitumor drugs also includes lung and colon cancers. Both Taxol® (paclitaxel) and Taxotere® (docetaxel) bind to tubulin, inhibit microtubule disassembly, and impair mitosis, thereby blocking progression through M phase of the cell cycle and facilitating apoptosis.

In spite of the undoubted overall clinical success of the taxoids, some tumors display resistance to these drugs. This drug resistance might be an innate feature of a tumor, or might develop in the tumor over time. Three main mechanisms of drug-resistance have been reported: (i) point mutations of the tubulin gene, (ii) selection of tubulin isoforms with low binding to taxanes, and (iii) expression of the multidrug-resistance (MDR) phenotype mediated by the P-glycoprotein (P-gp) efflux pump encoded by the mdr1 gene. Mechanism (iii) might explain the innate resistance to Taxol® and Taxotere® in tumors that often inherently express P-gp, such as colon and kidney cancers.

The preparation of Taxol®, Taxotere®, and their derivatives form the subject, for example, of European Patents EP 0,253,738 and EP 0,253,739 and International Application PCT WO 92/09,589.

It has now been found, and this forms the subject of the present invention, that the efficacy of Taxol®, Taxotere®, and their analogues can be considerably improved when they are administered in combination with at least one substance that is therapeutically useful in anti-cancer treatments and has a mechanism identical to or different from these taxanes.

A combination according to the invention includes a single composition comprising the recited components. It also includes multiple compositions that are used in the same administration regimen. For example, a combination can be a first composition comprising Taxotere® and a second composition comprising a cyclin-dependent kinase inhibitor, where both compositions are administered to a patient as part of a single therapeutic protocol. The combinations or associations according to the invention enable the phenomena of pleiotropic resistance or “multi-drug resistance” to be avoided or delayed.

Among substances that may be used in association or in combination with Taxol®, Taxotere®, or their analogues, there may be mentioned enzymes, such as L-asparaginase, and cyclin-dependent kinase inhibitors, such as flavopiridol, quercitin, and genistein. Various agents, such as biological response modifiers or growth factor inhibitors, such as interferons or interleukins, may also be used.

Because the activity of the products depends on the doses used, it is possible to use higher doses and to increase the activity while decreasing the toxicity phenomena or delaying their onset by combining growth factors of the haematopoietic type, such as G-CSF or GM-CSF or certain interleukins with Taxol®, Taxotere®, their analogues or their combinations with other therapeutically active substances.

Cyclin-dependent kinases (CDKs) are important regulators that control the timing and coordination of the cell cycle. CDKs form reversible complexes with their obligate cyclin partners to control transition through key junctures in the cell cycle. For example, the activated CDK4-cyclin D1 complex controls progression through the GI phase of the cell cycle, while the CDK1-cyclin B 1 complex controls entry into the mitotic phase of the cell cycle. Endogenous cyclin dependent kinase inhibitory proteins (CDKIs) are known that bind either the CDK or cyclin component and inhibit the kinase activity. In many tumors, such as melanomas and pancreatic and esophageal cancers, these natural CDKIs are either absent or mutated. Thus, selective CDK inhibitors might prove to be effective chemotherapeutic agents.

Flavopiridol (cis-5,7-dihydroxy-2-(2-chlorophenyl)-8-[4-(3-hydroxy-1-methyl)-piperidinyl]-1-benzopyran-4-one) is a synthetic flavone that has been shown to have antitumor activity against various tumor cells lines, such as human lung carcinoma and breast carcinoma. It also inhibits tumor growth in xenograft models. It has been shown to induce arrest in both the G1 and G2 phases of the cell cycle. Flavopiridol is a potent and selective inhibitor of the CDKs, and its antitumor activity is related to its CDK inhibitory activity. Studies have shown that its tumor cell growth inhibitory activity occurs in a cell cycle specific manner. See Bioorg. & Med. Chem. Letters 10:1037-1041(2000).

Thus, the present invention provides a combination that comprises: a) paclitaxel, docetaxel, or one or more derivatives of these, and b) at least one of: L-asparaginase, a cyclin-dependent kinase inhibitor, a biological response modifier, and a growth factor inhibitor. Accordingly, the invention includes combinations of Taxol®, Taxotere®, and their analogues with the cyclin-dependent kinase inhibitor, flavopiridol. For example, the combination can comprise docetaxel and flavopiridol.

Accordingly, the invention provides a pharmaceutical combination comprising: a) paclitaxel, docetaxel, or one or more derivatives of these, and b) a cyclin-dependent kinase inhibitor, in amounts such that the components of the combination provide therapeutic synergy in the treatment of at least one neoplastic disease, such as tumors and cancers. Included among these are breast cancer, lung cancer, and prostate cancer.

Taxotere® and flavopiridol have differing mechanisms, which can improve the efficacy of each. The improved efficacy of a combination according to the invention may be demonstrated by determination of the therapeutic synergy. A combination manifests therapeutic synergy if it is therapeutically superior to one or other of the constituents used at its optimum dose (T. H. Corbett et al, Cancer Treatment Reports 66:1187 (1982)).

To demonstrate the efficacy of a combination, it may be necessary to compare the maximum tolerated dose of the combination with the maximum tolerated dose of each of the separate constituents in the study in question. This efficacy may be quantified, for example, by the log₁₀ cell kill, which is determined according to the following formula:

log₁₀ cell kill=T-C (days)/3.32×T_{d}

in which T-C represents the time taken for the cells to grow, which is the mean time in days for the tumors of the treated group (T) and the tumors of the treated group (C) to have reached a predetermined value (1 g for example), and in which T_{d} represents the time in days needed for the volume of the tumor to double in the control animals (T. H. Corbett et al., Cancer 40:2660-2680 (1977); F. M. Schabel et al, “Cancer Drug Development, Part B”, Methods in Cancer Research 17:3-51, New York, Academic Press Inc. (1979)). A product is considered to be active if log₁₀ cell kill is greater than or equal to 0.7. A product is considered to be very active if log₁₀ cell kill is greater than 2.8.

The combination, used at its own maximum tolerated dose, in which each of the constituents will be present at a dose generally not exceeding its maximum tolerated dose, will manifest therapeutic synergy when the log₁₀ cell kill is greater than the value of the log₁₀ cell kill of the best constituent when it is administered alone.

Thus, the present invention provides a method of treating a neoplastic disease. The method comprises administering a combination comprising the following components: a) paclitaxel, docetaxel, or one or more derivatives of these, and b) at least one of: L-asparaginase, a cyclin-dependent kinase inhibitor, a biological response modifier, and a growth factor inhibitor; to a subject in an amount sufficient to treat a neoplastic disease. The components can be administered together or they can be administered separately, for example, at different times. The method can be used to treat neoplastic diseases such as breast cancer, lung cancer, and prostate cancer. In embodiments, the combination comprises docetaxel and flavopiridol.

Various treatment cycles and regimens can be used. The particular regimen and cycle can be determined by those of skill in the art according to standard protocols and without undue or excessive experimentation. For example, a treatment cycle can include a ten day treatment cycle in which docetaxel is administered on the first and last days and flavopiridol is administered on the first four days and last four days of the ten day cycle. Likewise, a treatment cycle can include a 23 day treatment cycle in which docetaxel is administered on days 14 and 23 and flavopiridol is administered on days 14 through 17 and days 20 through 23. Similarly, a treatment cycle can include a 25 day treatment cycle in which docetaxel is administered on days 14 and 25 and flavopiridol is administered orally on days 14 through 18 and days 21 through 25.

The method can comprise administration of the combination more than one time. Thus, the method can comprise a treatment regimen that includes a series of more than one administration of the combination.

The efficacy of the combinations on solid tumors may be determined experimentally in the following manner:

The animals subjected to the experiment, generally mice, are subcutaneously grafted bilaterally with 30 to 60 mg of a tumor fragment on day zero. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor. The animals are observed every day. The different animal groups are weighed 3 or 4 times a week until the maximum weight loss is attained, and the groups are then weighed at least once a week until the end of the trial.

The tumors are measured 2 or 3 times a week until the tumor reaches approximately 2 g, or until the animal dies, if this occurs before the tumor reaches 2 g. The animals are autopsied after death.

The antitumor activity is determined in accordance with the different parameters recorded in Tables I and II.

For a study of the combinations on leukemias, the animals are grafted with a particular number of cells, and the antitumor activity is determined by the increase in the survival time of the treated mice relative to the controls. The product is considered to be active if the increase in survival time is greater than 27%, and is considered to be very active if the increase in survival time is greater than 75%, in the case of P388 leukemias.

In the Examples that follow, mice were grafted with mammary adenocarcinoma MA 13/C and treated with combinations of Taxotere® and flavopiridol, using different schedules of administration and different modes of administration. Some of these schedules show clear therapeutic synergy. All acceptable modes of administration are contemplated by the invention. For example, flavopiridol may be administered orally as well as intravenously.

Additional objects and advantages of the invention will be set forth in part in the description that follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention as claimed.

### EXAMPLE 1

In order to determine the activity of each constituent of the combination, Taxotere® and flavopiridol were given alone to MA 13/C bearing mice. Taxotere® was administered intravenously on days 15 and 21; the dose on each day was 30 mg/kg for a total dosage of 60 mg/kg. Used alone, this dosage resulted in a log₁₀ cell kill of 4.7 and a complete response in all 5 of the mice so treated. Administration of flavopiridol alone on days 15 and 21 in a dose of 6 mg/kg each day, or a total dosage of 12 mg/kg, resulted in no complete responses in the 5 mice tested.

The compounds were then combined in two ways and tested in an intermittent schedule. In the first combination, flavopiridol was given first on days 15 and 20 (total dosage 9 mg/kg) and Taxotere® was given on the following days 16 and 21 (total dosage 45 mg/kg). When given in this fashion, the log₁₀ cell kill was 5.5 and resulted in 5 of 5 complete responses in the mice treated. In this intermittent schedule, the log₁₀ cell kill was better than Taxotere® given alone and still resulted in 5 of 5 complete responses despite the fact that 25% less Taxotere® and 25% less flavopiridol were administered in this combination than in the control.

When the constituents of the combination were reversed and Taxotere® was given first on days 15 and 20 and flavopiridol was given on days 16 and 21 in the same amounts, the log₁₀ cell kill was slightly less than Taxotere® alone, but again there were 5 of 5 complete responses. Table I illustrates these results and shows that the combination of flavopiridol and Taxotere® administered in 25% lesser amounts had an efficacy similar to Taxotere® alone.

TABLE I
Flavopiridol- Taxotere ® Combination in
MA13/C bearing mice: 24 hours apart

| | | HNTD (DT) | | | | |
|---|---|---|---|---|---|---|
| | | mg/kg | | % bwl | | Com- |
| IV Agent | Schedule | Docetaxel | Flavo | (nadir) | lck | ments |
| Taxotere ® | 15, 21 | 30.0 | — | 8 (27) | 4.7 | 5/5 CR |
| | | (60.0) | | | | HDE |
| Flavopiridol | 15, 21 | — | 6.0 | 6 (16) | 0.4 | 0/5 CR |
| | | | (12.0) | | | |
| Combination | | | | | | |
| 24 h apart | | | | | | |
| Flavo 1^{st} | 15, 20 | 22.5 | 4.5 | 7 (22) | 5.5 | 5/5 CR |
| Taxotere ® | 16, 21 | (45.0) | (9.0) | | | HDE |
| Taxotere ® 1^{st} | | 22.5 | 4.5 | 7 (25) | 4.4 | 5/5 CR |
| | | (45.0) | (9.0) | | | HDE |

Td = 2.2 days; time for control to reach 1 g = 20.3 days; median tumor burden at start of therapy 130-160 mg; CR = complete response; IV = intravenous; bwl = body weight loss at nadir; (DT) = total dose; HNTD = highest non-toxic dose; lck = log₁₀ cell kill.

### EXAMPLE 2

The number of days on which flavopiridol was administered were increased. It was discovered that when the MA 13/C bearing mice were exposed to similar doses of flavopiridol given over 8 days in a 10 day cycle, and when Taxotere® was given on the first and last day of the ten day cycle, there was clear synergy.

Table II gives the highest non-toxic total dose of each component alone: 96.8 mg/kg of Taxotere® and 23.2 mg/kg of flavopiridol. When the constituents were administered in combination, with Taxotere® being administered on days 14 and 23 and flavopiridol on days 14-17 and 20-23, three combinations were clearly synergistic and a fourth was equal in efficacy to Taxotere® alone. All four combinations resulted in 6 of 6 complete responses; i.e., 100% complete responses.

TABLE II
Flavopiridol-Taxotere ® Combination
Mammary adenocarcinoma MA13/C << repeated exposure >>

| IV agents mg/kg/dose | | | | | |
|---|---|---|---|---|---|
| (total dose) | | | | | |
| Taxotere ® | Flavopiridol | % bwl | lck | | |
| day 14, 23 | day 14-17, 20-23 | (nadir) | Gross | CR | Comments |
| 78.1 (156.2) | — | 20 (29) | — | — | 2/6 DD |
| 48.1 (96.8) | — | 8 (28) | 4.4 | 6/6 | HNTD |
| — | 4.8 (38.4) | >20 | — | — | 4/5 DD |
| — | 2.9 (23.2) | 3 (18) | 1.0 | 0/6 | HNTD |
| — | 1.75 (14) | +10 (24) | 0.0 | 0/6 | |
| 48.4 (96.8) | 2.9 (23.2) | 15 (27) | — | — | 2/5 DD |
| 53.2 (106.4) | 1.93 (15.44) | 13 (29) | 9.0 | 6/6 | HNTD |
| 43.6 (87.2) | 1.6 (12.8) | 9 (29) | 7.0 | 6/6 | |
| 36.3 (72.6) | 1.31 (10.48) | 3 (29) | 5.1 | 6/6 | |
| 31.5 (63.0) | 1.14 (9.12) | 6 (28) | 4.4 | 6/6 | |
| 24.2 (48.4) | 0.88 (7.04) | 5 (19) | 2.0 | 2/6 | |

Td = 2.2 days. Time for control to reach 1 g = 17.8 days; median tumor burden at start of therapy 210-260 mg.; CR = complete response; IV = intravenous; bwl = body weight loss at nadir; (DT) = total dose; HNTD = highest non-toxic dose; lck = log₁₀ cell kill.

A 3-arm dose-response study was performed in C3H/HeN mice bearing measurable tumors at start of therapy (230 mg). The model chosen was a murine mammary adenocarcinoma MA13/C, selected on the basis of its chemosensitivity to docetaxel. Mice were treated with Flavo (i.e., flavopiridol once a day, day 14-17, and day 20-23 post tumor implantation), or docetaxel (i.e., on days 14 and 23), or their combination.

Results:

At the highest non-toxic dose (HNTD, 2.9 mg/kg/dose, total dose of 23.2 mg/kg), Flavo administered IV as a single agent was inactive with a −0.4 log cell kill net (log cell kill net=tumor growth delay−treatment duration/3.32×tumor doubling time), and no complete regression (CR). The HNTD of docetaxel alone (48.1 mg/kg/injection, total dose of 96.8 mg/kg) was found very active (3 log cell kill net, 6/6 CR). Clear synergy was obtained at the highest non-toxic combination (Flavo at 1.93 mg/kg/dose and docetaxel at 53.2 mg/kg/injection) with a 7.6 log cell kill net and 6/6 CR. This combination was well tolerated inducing a 13% body weight loss at nadir occurring 6 days post last treatment. Synergy was retained on 2 additional lower dose levels compared to docetaxel HNTD. This synergy was also observed when Flavo was administered orally.

### EXAMPLE 3

Taxotere® was injected IV on days 14 and 25 post tumor implantation. Flavo was orally administered once a day from day 14 to 18 and from day 21 to 25.

TABLE III
FLAVOPIRIDOL (po) - DOCETAXEL (iv)
Prolonged exposure MA13/C

| | | HNTD (DT) | | | | |
|---|---|---|---|---|---|---|
| | | mg/kg | | % bwl | | |
| Agent | Schedule | TXT | Flavo | (nadir) | lck | Comments |
| Taxotere ® | 14, 25 | 30 | — | 3.6 (33) | 0.9 | HNTD |
| | | (60) | | | | |
| Flavopiridol | 14-18, 21-25 | | 2.7 | 11.6 (26) | 0.1 | HNTD |
| | | | (27) | | | |
| Combination | 14, 25 | 45 | | 9.7 (19) | ≅5 | HNTC |
| Taxotere ® | | (90) | | | | |
| Combination | 14-18, 21-25 | | 4.5 | | | |
| Flavopiridol | | | (45) | | | |
| Combination | 14, 25 | 45 | | 9.3 | 3.2 | HNTC |
| Taxotere ® | | (90) | | | | |
| Combination | 14-18, 21-25 | | 2.0 | | | |
| Flavopiridol | | | (20) | | | |

HNTD = highest non-toxic dose; (DT) = total dose; bwl = body weight loss at nadir; lck = log₁₀ cell kill; TXT = Taxotere ®; Flavo = flavopiridol; HNTC = highest non-toxic combination. ≅320 mg - tumor weight on day 14.

As in Example 2, the combination of docetaxel and repeated daily Flavo was found more active than either of the single agents, at equitoxic dosages. Synergy is shown by the log 4 increase in cells killed.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A combination comprising: a) paclitaxel, docetaxel, or one or more derivatives thereof, and b) at least one of: L-asparaginase, a cyclin-dependent kinase inhibitor, a biological response modifier, and a growth factor inhibitor.

2. The combination of claim 1, wherein said combination comprises paclitaxel.

3. The combination of claim 1, wherein said combination comprises docetaxel.

4. The combination of claim 1, wherein said combination comprises L-asparaginase.

5. The combination of claim 1, wherein said combination comprises a cyclin dependent kinase inhibitor.

6. The combination of claim 5, wherein said cyclin dependent kinase inhibitor is flavopiridol.

7. The combination of claim 5, wherein said cyclin de pendent kinase inhibitor is quercitin or genistein.

8. The combination of claim 1, comprising docetaxel and flavopiridol.

9. The combination of claim 1, wherein the combination is a single composition.

10. A pharmaceutical combination comprising: a) paclitaxel, docetaxel, or one or more derivatives thereof, and b) a cyclin-dependent kinase inhibitor, in amounts sufficient to provide therapeutic synergy in the treatment of at least one neoplastic disease.

11. The pharmaceutical combination of claim 10, wherein the neoplastic disease is breast cancer.

12. The pharmaceutical combination of claim 10, wherein the neoplastic disease is lung cancer.

13. The pharmaceutical combination of claim 10, wherein said combination comprises paclitaxel.

14. The pharmaceutical composition of claim 10, wherein said combination comprises docetaxel.

15. The pharmaceutical combination of claim 10, wherein said cyclin-dependent kinase inhibitor is flavopiridol.

16. A method of treating a neoplastic disease, said method comprising administering a combination comprising the following components:
a) paclitaxel, docetaxel, or one or more derivatives thereof, and
b) a cyclin-dependent kinase inhibitor, a biological response modifier, to a subject in an amount sufficient to treat said neoplastic disease.

17. The method of claim 16, wherein said components are administered separately.

18. The method of claim 17, wherein said components are administered at different times.

19. The method of claim 17, wherein the neoplastic disease is breast cancer.

20. The method of claim 17, wherein the neoplastic disease is lung cancer.

21. The method of claim 17, wherein said cyclin dependent kinase inhibitor is flavopiridol.

22. The method of claim 21, wherein said flavopiridol is administered orally.

23. The method of claim 17, wherein the combination comprises docetaxel and flavopiridol.

24. The method of claim 23, wherein the method comprises a ten day treatment cycle in which docetaxel is administered on the first and last days and flavopiridol is administered on the first four days and last four days of said ten day cycle.

25. The method of claim 23, wherein the method comprises a 23 day treatment cycle in which docetaxel is administered on days 14 and 23 and flavopiridol is administered on days 14 through 17 and days 20 through 23.

26. The method of claim 23, wherein the method comprises a 25 day treatment cycle in which docetaxel is administered on days 14 and 25 and flavopiridol is administered orally on days 14 through 18 and days 21 through 25.
